(19) United States
(12) Patent Application Publication
(10) Pub. No.: US 2018/0221417 A1
(43) Pub. Date: Aug. 9, 2018

(54) TREATMENT OF DRY EYE USING AMNION RELEASED FACTORS

(71) Applicant: Aidan Research and Consulting, LLC, Westlake, TX (US)

(72) Inventor: Neil H. Riordan, West Lake, TX (US)

(73) Assignee: Aidan Research and Consulting, LLC, Westlake, TX (US)

(21) Appl. No.: 15/891,307

(22) Filed: Feb. 7, 2018

Related U.S. Application Data
(60) Provisional application No. 62/456,102, filed on Feb. 7, 2017.

(51) Int. Cl. A61K 35/50; A61P 27/02; A61K 9/00; A61K 45/06

(52) U.S. Cl. CPC A61K 35/50; A61K 45/06; A61K 9/0048; A61P 27/02

(57) ABSTRACT

Disclosed are means, methods and compositions of matter for treatment of dry eye disease using factors released from placental amniotic tissue when cultured in a liquid media. In one embodiment of the invention amnion is extracted from a hemochorial placenta and cultured in a means to maintain viability of said amnion. Said means in which hemochorial placenta amnion is maintained viable is subsequently used to collect factors which are released by said placental amnion which are useful for treatment of dry eye disease either in unpurified form, in concentrated form, or specific molecular weight fractions derived thereof.

### CROSS-REFERENCE TO RELATED APPLICATIONS

This application claims priority to U.S. Provisional Application No. 62/456,102, filed Feb. 7, 2017, which is incorporated herein by reference in its entirety

### FIELD OF THE INVENTION

The invention pertains to the field of ophthalmology, more specifically the invention pertains to treatment of dry eye. More specifically the invention provides means of treating dry eye by administration of liquid media, or formulated liquid media that has been in contact with amniotic tissue.

### BACKGROUND OF THE INVENTION

Over 20 million adults in the United States are afflicted with dry eye syndrome (DES), also known as keratoconjunctivitis sicca (KCS) or keratitis sicca.

The proper function of the eye depends on the flow of tears to provide constant moisture and lubrication to maintain vision and comfort. Tears are a combination of water, for moisture; oils, for lubrication; mucus, for even spreading; and antibodies and special proteins, for resistance to infection. These components are secreted by special glands located around the eye. When there is an imbalance in this tear system, a person may experience dry eyes. This irritating and painful condition has symptoms ranging from itching, burning, tearing, and blurred vision to devastating sequelae including corneal scarring and permanent vision loss. Symptoms include eye redness, a yellow or greenish discharge, ulceration of the cornea, pigmented cornea, and blood vessels on the cornea. Diagnosis is made by measuring tear production with a Schirmer tear test. Less than 15 millimeters of tears produced in a minute is abnormal. DES may be subdivided into 2 main types: DES associated with Sjogren syndrome (SS) and DES unassociated with SS (non-SS KCS). Its prognosis shows considerable variance, depending upon the severity of the condition. Most patients have mild-to-moderate cases, and can be treated symptomatically with wetting and lubricating eye drops.

Blepharitis, chronic eyelid inflammation, is one of the leading causes of evaporative dry eye, and it is seen in nearly one-half of ophthalmology visits. Recent studies have linked blepharitis to other chronic inflammatory/autoimmune conditions, including gastritis, peptic ulcer disease, asthma, and inflammatory bowel disease. While persons with autoimmune diseases have a high likelihood of having dry eyes, most persons with dry eyes do not have an autoimmune disease. Instances of Sjogren syndrome associated with DES are present much more commonly in women, with a ratio of 9:1. Milder forms of DES are also more common in women, partly because hormonal changes, such as those that occur in pregnancy, menstruation, and menopause, can decrease tear production. In areas of the world where malnutrition is common, vitamin A deficiency is a common cause of DES. Racial predilections do not exist for this disease.

To date, treatment of dry eye syndrome has remained elusive, and consists primarily of the supportive care of artificial tears, lid scrubs, and warm compresses. Corticosteroids and cyclosporine are partially effective in reducing the clinical signs and symptoms of DES but have significant ocular complications such as cataract formation and elevated intraocular pressure leading to glaucoma. Topical cyclosporin (topical cyclosporin A, tCSA) 0.05% ophthalmic emulsion is an immunosuppressant, marketed in the United States by Allergan under the trade name Restasis®. Restatis®, approved by the U.S. Food and Drug Administration in 2002, decreases surface inflammation presumably through inhibition of transcription factors required for cytokine production and T-lymphocyte maturation. Other treatments include temporary punctal occlusion which involves closing the ducts that drain tears out of the eye. This may be done temporarily with a dissolving plug that is inserted into the tear drain of the lower eyelid to determine whether permanent plugs can provide an adequate supply of tears. Non-dissolving punctal plugs and punctal occlusion by cautery (application of heat to tear exit duct) is often done if temporary plugging of the tear drains works well. These measures increase the tear level by blocking the “drainpipe” through which tears normally exit the eye and enter the nose. The plugs can be easily removed. More details on DES and blepharitis are presented below.

### SUMMARY

Embodiments herein are directed to the following embodiments:

1) A method of treating dry eye disease comprising:
a) identifying a patient suffering from dry eye disease;
b) obtaining a hemochorial placenta;
c) isolating the amnion from said hemochorial placenta;
d) contacting said amnion with a liquid media in a manner to preserve viability of cellular components of said amnion;
e) applying said contacted liquid media, or portions thereof, to said patient in an amount to treat or ameliorate the effects of dry eye disease.
   2. The method of claim 1, wherein said dry eye disease is associated with reduced lacrimal secretions.
   3. The method of claim 1, wherein said dry eye disease is associated with hyperosmolarity of lacrimal secretions.
   4. The method of claim 1, wherein said dry eye disease is associated with keratitis.
   5. The method of claim 1, wherein said dry eye disease is associated with production of inflammatory cytokines.
   6. The method of claim 5, wherein said inflammatory cytokines are selected from the group consisting of: a) IL-1 beta; b) IL-6; c) TNF-alpha; and d) IL-15
   7. The method of claim 1, wherein said dry eye disease is associated with Meibomian gland dysfunction.
   8. The method of claim 1, wherein said liquid media subsequent to contact with said amnion is endowed with one or more ingredients selected from factors and agents that promote any one or more of survival, health, cell attachment and normal differentiation of ocular surface epithelial cells and optionally factors and agents that prevent squamous metaplasia; one or more agents capable of altering the fluid properties of a tear film including at least one agent capable of establishing or maintaining a stable tear film and optionally one or more agents selected from the group consisting of opthalmological lubricating agents, viscosity enhancing agents and agents capable of reducing tear film evaporation.
   9. The method of claim 1 wherein said amnion is contacted with said liquid media by means of immersion of said amnion in said liquid media.
   10. The method of claim 1, wherein said amnion is cultured in said liquid media at 37 Celsius in a fully humidified atmosphere.
   11. The method of claim 1, wherein said liquid media is concentrated and subsequently desalted.
   12. The method of claim 1, wherein said liquid media is concentrated by filtration and subsequently desalted.
   13. The method of claim 1, wherein said liquid media is concentrated by lyophilization and subsequently desalted.
   14. The method of claim 1, wherein said liquid media is used as a source for identifying factors capable of inhibiting dry eye disease.
   15. The method of claim 1, wherein said liquid media is applied to the ocular surface.
   16. The method of claim 1, wherein said liquid media is used to formulate an ophthalmic composition.
   17. The method of claim 16, wherein said ophthalmic composition comprises a bacteriological preservative.
   18. The method of claim 17, wherein the bacteriological preservative is selected from the group consisting of benzalkonium chloride, thimerosal, phenylmercuric nitrate, chlorobutanol, and sorbicacid.
   19. The method of claim 17, wherein the ophthalmic composition further comprises a chelating agent.
   20. The method of claim 17, wherein the ophthalmic composition further comprises an antibiotic.

### DESCRIPTION OF THE INVENTION

Unless defined otherwise, technical and scientific terms used herein have the same meaning as commonly understood by one of ordinary skill in the art to which this invention belongs. The practice of the present invention will employ, unless indicated specifically to the contrary, conventional methods of molecular biology and recombinant DNA techniques within the skill of the art, many of which are described below for the purpose of illustration. Such techniques are fully explained in the literature. See, e.g., Singleton et al., Dictionary of Microbiology and Molecular Biology 3rd.sup.ed., J. Wiley & Sons (2001); March, Advanced Organic Chemistry Reactions, Mechanisms and Structure 5th.sup.ed., J. Wiley & Sons (2001); Sambrook & Russell, eds., Molecular Cloning: A Laboratory Manual 3rd ed., Cold Spring Harbor Laboratory Press (2001); Glover, ed., DNA Cloning: A Practical Approach, vol. I & II (2002); Gait, ed., Oligonucleotide Synthesis: A practical approach, Oxford University Press (1984); Herdewijn, ed., Oligonucleotide Synthesis: Methods and Applications, Humana Press (2005); Hames & Higgins, eds., Nucleic Acid Hybridisation: A Practical Approach, IRL Press (1985); Buzdin & Lukyanov, eds., Nucleic Acid Hybridization: Modern Applications, Springer (2007); Hames & Higgins, eds., Transcription and Translation: A Practical Approach, IRL Press (1984); Freshney, ed., Animal Cell Culture, Oxford UP (1986); Freshney, Culture of Animal Cells: A Manual of Basic Technique and Specialized Applications, 6th ed., John Wiley & Sons (2010); Perbal, A Practical Guide to Molecular Cloning, 3rd ed., Wiley-Liss (2014); Farrell, RNA Methodologies: A Laboratory Guide for Isolation and Characterization, 3rd ed., Elsevier/Focal Press (2005); Lilley & Dahlberg, eds., Methods in Enzymology: DNA Structures, Part A: Synthesis and Physical Analysis of DNA, Academic Press (1992); Harlow & Lane, Using Antibodies: A Laboratory Manual: Portable Protocol no. 1, Cold Spring Harbor Laboratory Press (1999); Harlow & Lane, eds., Antibodies: A Laboratory Manual, Cold Spring Harbor Laboratory Press (1988); Seethala & Fernandes, eds., Handbook of Drug Screening, Marcel Dekker (2001); and Roskams & Rodgers, eds., Lab Ref: A Handbook of Recipes, Reagents, and Other Reference Tools for Use at the Bench, Cold Spring Harbor Laboratory (2002), which provide one skilled in the art with a general guide to many of the terms used in the present application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The present invention provides a method of treating dry eye by topically administering to the eye a liquid composition, wherein said liquid composition has been in contact with placental amnion.

The compositions of this invention protect the ocular surface from dryness and absorb shear forces of the blink, and assist the eye's own secreted gel forming mucins (predominantly MUCS) in maintaining their viscoelastic properties and ensuring structure and stability of the tear film, thereby slowing or preventing the changes to the ocular surface seen in dry eye conditions. In one embodiment, a method for treating an epithelial lesion of the eye or ophthalmic disorder is disclosed including topically administering an ophthalmic composition including media that has been in contact with placental amnion, or concentrate or cell-free extract of the media, alone or in a combination with a pharmaceutically acceptable carrier to the ocular surface or immediate vicinity of an eye of a subject in need thereof. In one related aspect, the ophthalmic disorder is dry eye or keratonconjunctivitis sicca (KCS). In another related aspect, the subject has an epithelial lesion of the eye. In one aspect, the composition includes one or more human growth factors. In a related aspect, the one or more growth factors are selected from EGF or TGF-.beta. or combinations thereof. In a further related aspect, the one or more growth factors include GM-CSF, IL-15, IL-1a, IL-2, IL-4, IL-5, IL-6, IL-7, IL-8, MCP-1, TNF.alpha., FGF-2, Flt-3, PDGF-AA, TGF-.beta.1, TGF-.beta.2, and TGF-.beta.3, or combinations thereof. In another aspect, the composition further includes retinol. In one embodiment of the invention dry eye is associated with a disease or disorder selected from the group comprising dry eye syndrome, blepharitis, meibomitis, ocular rosacea, thyroid eye disease, chronic papillary conjunctivitis, chronic follicular conjunctivitis, nonspecific chronic conjunctivitis, giant papillary conjunctivitis, ocular cicatricial pemphigoid, cicatrizing conjunctivitis, allergic conjunctivitis, phlyctenular corneoconjunctivitis, episcleritis, diffuse scleritis, nodular scleritis, scleromalacia perforans, superficial punctate keratitis, infectious keratitis, peripheral ulcerative keratitis, Thygeson's superficial punctate keratitis, corneal graft rejection, disciform keratitis, stromal keratitis, anterior uveitis, HLA-B27 uveitis, Parkinson's disease, Behcet's disease, atopic eye disease, pars planitis, sarcoidosis, sympathetic ophthalmia, Fuch's heterochromic iridocyclitis, glaucomatocyclitis crisis (Posner-Schlossman syndrome), chronic macular edema, cystoid macula edema, central serous choreoretinopathy, white dot syndrome, anemia, hypertension, stroke, mumps, acute retinal necrosis, and autoimmune diseases comprising juvenile rheumatoid arthritis, lupus erythematosus, cystic fibrosis, Sjogren's syndrome, Vogt-Koyanagi-Harada syndrome, HIV/AIDS, and diabetes.

The therapeutic agents of the present invention (i.e., liquid media that has been in contact with placental amnion) may be administered to a human or animal subject by known procedures, preferably topically to the eye. The therapeutic agents can be administered separately or in a single formulation. For example, an amount of the media that has been in contact with the amnion may be packaged in a vial or unit dose. The dosage or therapeutically effective amount of the therapeutic media in a formulation will depend on the concentration of the active therapeutic agent in the formulation, the amount of the formulation administered (i.e. how may eye drops are used) and on its rate of absorption, inactivation, and excretion. It is to be noted that dosage values may also vary with the severity of the condition to be alleviated. It is to be further understood that for any particular subject, specific dosage regimens should be adjusted over time according to the individual's need and the professional judgment of the person administering or supervising the administration of the compositions. Typically, dosing will be determined using techniques known to one skilled in the art.

The dosage/amount of therapeutic agent administered may vary depending on the symptoms, age and other physical characteristics of the patient, the nature and severity of the disorder to be treated or prevented, the degree of comfort desired, the route of administration, and the concentration of the therapeutic agent in the formulation. Any of the pharmaceutical formulations described herein may be administered in a single dose or in divided doses.

An effective dose or amount, and any possible effects on the timing of administration, may need to be identified for any particular formulation (for example, based on concentration of the active agent) or method of the present invention. This may be accomplished by routine experiment as described herein. The effectiveness of any formulation and method of treatment or prevention may be assessed by administering the formulation and assessing the effect of the administration by measuring one or more indices or symptoms associated with dry eye to determine that the formulation reduces one or more of the indices or symptoms, and with the degree of comfort to the patient, compared to pretreatment indices or symptoms, or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of the active agents, the physiological condition of the patient (including age, sex, disease type and stage, general physical condition, responsiveness to a given dosage and type of medication), the route of administration, and the like. Guidelines may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices of ocular inflammation at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of one week from the onset of therapy, and subsequent reevaluations occurring periodically every few days or every one to two weeks during therapy and then every month thereafter. Adjustments to the amount(s) of the therapeutic agents administered and possibly to the time of administration may be made based on these reevaluations. Treatment may be initiated with smaller dosages which are less than the optimum dose of the compound. Thereafter, the dosage may be increased by small increments until the optimum therapeutic effect is attained. Toxicity and therapeutic efficacy may be determined by standard pharmaceutical procedures in cell cultures or experimental animals, e.g., for determining the LD.sub.50 and the ED.sub.50. Topical formulations have lower risk of toxicity than systemically administered formulations. The pharmaceutical compositions described above may additionally comprise one or more additional active ingredients defined herein to include tear substitutes, antiallergenic agents and vasoconstrictors. Such compositions may be used to treat or prevent dry eye or dry mouth or both, and they can also be used to treat an underlying or concurrent disorder or disease such as ocular allergy or other autoimmune inflammatory eye diseases or to treat or prevent symptoms accompanying dry eye/dry mouth. For example, an embodiment of a pharmaceutical composition may comprise the therapeutic media and a tear substitute or other lubricant. Or, it may comprise a media that has been endowed with therapeutic properties after having been in contact with placental amnion and an antiallergenic agent, and optionally a tear substitute or lubricant.

Exemplary uses of various agents in treating ocular allergy and compositions thereof are described in U.S. patent application Ser. No. 10/762,201 filed Mar. 20, 2004 and U.S. patent application Ser. No. 11/074,000, filed Mar. 3, 2005, which claims priority to U.S. Provisional Patent Application 60/549,703, filed Mar. 3, 2004, all of which applications are incorporated by reference in their entireties. The dosages and combinations of agents described therein, for example, may be combined with the presently described formulations. A variety of tear substitutes are known in the art and include, but are not limited to: monomeric polyols (such as glycerol, propylene glycol, and ethylene glycol); polymeric polyols (such as polyethylene glycol); cellulose esters (such as hydroxypropylmethyl cellulose, carboxy methylcellulose sodium and hydroxy propylcellulose); dextrans (such as dextran 70); water soluble proteins (such as gelatin); vinyl polymers (such as polyvinyl alcohol, polyvinylpyrrolidone, and povidone); and carbomers (such as carbomer 934P, carbomer 941, carbomer 940 and carbomer 974P). Many such tear substitutes are commercially available, which include, but are not limited to, cellulose esters such as Bion Tears®, Celluvisc®, Genteal®, OccuCoat®, Refresh®, Teargen II®, Tears Naturale®, Tears Naturale II®, Tears Naturale Free®, and TheraTears®; and polyvinyl alcohols such as Akwa Tears® HypoTears®, Moisture Eyes®, Murine Lubricating®, and Visine Tears®. Tear substitutes may also be comprised of paraffins, such as the commercially available Lacri-Lube ointments. Other commercially available ointments that are used as tear substitutes include Lubrifresh PM®, Moisture Eyes PM® and Refresh PM®.

Exemplary antihistamines include, but are not limited to, pheniramine, emedastine difumarate and levocabastine. In other embodiments, the invention features pharmaceutical compositions comprising an effective amount of a mast cell stabilizer and a tear substitute. Exemplary mast cell stabilizers include, but are not limited to, nedocromil, lodoxamide, cromolyn, and cromolyn sodium. Exemplary drugs with multiple modes of action include, but are not limited to, azelastine, epinastine, olopatadine and ketotifen fumarate. Exemplary vasoconstrictors include, but are not limited to, naphazoline, antolazine, tetrahydozoline and oxymetazoline.

The therapeutic agents in the pharmaceutical formulations and any additional active agents such as antiallergenic agents or other active ingredients may be in the form of a pharmaceutically acceptable salt. In some embodiments the pharmaceutical compositions will be formulated as solutions, suspensions, or ointments and other dosage forms for topical ophthalmic or oral administration in a pharmaceutically acceptable carrier, adjuvant, or vehicle. Aqueous solutions are generally preferred, based on ease of formulation, as well as a patient's ability to easily administer such compositions by means of instilling one to two drops of the solutions in the affected eyes or used as an eye wash. For dry mouth formulations may be sprayed in the mouth or used as a mouth wash. However, the compositions may also be suspensions, viscous or semi-viscous gels, or other types of solid or semi-solid compositions.

Any of a variety of carriers may be used in the topical formulations of the present invention, including water, mixtures of water and water-miscible solvents (such as C.sub.1- to C.sub.7-alkanols), vegetable oils or mineral oils comprising from 0.5 to 5% non-toxic water-soluble polymers, natural products (such as gelatin, alginates, pectins, tragacanth, karaya gum, xanthan gum, carrageenin, agar and acacia), starch derivatives (such as starch acetate and hydroxypropyl starch), and also other synthetic products (such as polyvinyl alcohol, polyvinylpyrrolidone, polyvinyl methyl ether, or polyethylene oxide—preferably cross-linked polyacrylic acid, such as neutral Carbopol), or mixtures of those polymers. The concentration of the carrier is, typically, from 1 to 100,000 times the concentration of the active ingredient.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ or portion of the body, to another organ or portion of the body. Each carrier must be “acceptable” in the sense of being compatible with the other ingredients of the supplement and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

Additional non-active ingredients that may be included in the formulations include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0 or 6.5 to 7.8. Suitable buffers may be added, such as boric acid, sodium borate, potassium citrate, citric acid, sodium bicarbonate, TRIS, and various mixed phosphate buffers (including combinations of Na.sub.2HPO.sub.4, NaH.sub.2PO.sub.4 and KH.sub.2PO.sub.4) and mixtures thereof. Borate buffers are preferred. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example, be of ionic or non-ionic type. Examples of ionic tonicity enhancers are alkali metal or earth metal halides, such as, for example, CaCl.sub.2, KBr, KCl, LiCl, Nal, NaBr or NaCl, Na.sub.2SO.sub.4 or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the topical formulations additionally comprise a preservative. A preservative may typically be selected from a quaternary ammonium compound such as benzalkonium chloride, benzoxonium chloride or the like. Benzalkonium chloride is better described as N-benzyl-N-(C.sub.8-C.sub.18 alkyl)-N,N-dimethylammonium chloride. Examples of preservatives different from quaternary ammonium salts are alkyl-mercury salts of thiosalicylic acid, such as, for example, thiomersal (thimerosol), phenylmercuric nitrate, phenylmercuric acetate or phenylmercuric borate; sodium perborate; sodium chlorite; parabens, such as, for example, methylparaben or propylparaben; alcohols, such as, for example, chlorobutanol, benzyl alcohol or phenyl ethanol; guanidine derivatives, such as, for example, chlorohexidine or polyhexamethylene biguanide; sodium perborate; or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements. Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family, vinyl polymers, and acrylic acid polymers.

### Example

A clinical trial was performed in which 9 patients were administered eye drops comprising of media that had been in contact with placental amnion. Said media was used as eyedrops and administered as indicated below. Assessment was made for the following characteristics: a) Ocular Surface Disease Index (OSDI) questionnaire; b) Shirmers test; c) Tear Osmolarity; d) Corneal Staining; e) Visual Acuity; and f) Artificial Tears used per day. Improvements were observed in the majority of patients. Results are shown in Table 1 below.

| 001-F-S | Visit 1 | | Visit2 | | Visit 3 | | Visit 4 | |
|---|---|---|---|---|---|---|---|---|
| DOSING BID | | | | | | | | |
| OSDI | 25 | | 2.1 | | 8.3 | | 10.4 | |
| | OD | OS | OD | OS | OD | OS | OD | OS |
| SHIRMERS | 21 | 25 | 20 | 16 | 13 | 12 | 11 | 10 |
| TEAR OSMOLARITY | 282 | 278 | 288 | 291 | 283 | 282 | 281 | 298 |
| CORNEAL STAINING | 9 | 6 | 6 | 5 | 4 | 4 | 2 | 2 |
| VISUAL ACUITY | 20/25 | 20/20 | 20/25 + 2 | 20/30 | 20/25 | 20/30 | 20/20 − 2 | 20/20 − 1 |
| ARTIFICIAL TEARS USED PER DAY | | | V.2 QID OU | | V.3 0-Qday OU | | V.4 Zero | |
| | | | | | | | tears used | |
| 002-BMP | Visit 1 | | Visit 2 | | Visit 3 | | Visit 4 | |
| DOSING BID | | | | | | | | |
| OSDI | 36.1 | | 33.3 | | 15 | | 12.5 | |
| | OD | OS | OD | OS | OD | OS | OD | OS |
| SHIRMERS | 9 | 20 | 7 | 13 | 8 | 10 | 7 | 6 |
| TEAR OSMOLARITY | 296 | 302 | 277 | 276 | 305 | 300 | 300 | 301 |
| CORNEAL STAINING | 7 | 8 | 6 | 6 | 6 | 6 | 5 | 5 |
| VISUAL ACUITY | 20/30 | 20/40 | 20/25 + 2 | 20/20 | 20/25 − 1 | 20/20 | 20/25 | 20/20 − 2 |
| ARTIFICIAL TEARS USED PER DAY | | V.2 Zero | | | V.3 Zero | | V.4 Zero | |
| | | tears used | | | tears used | | tears used | |
| 003-JKK | Visit 1 | | Visit 2 | | Visit 3 | | Visit 4 | |
| DOSING DAY | | | | | | | | |
| OSDI | 43.2 | | 22.5 | | 38.6 | | 40.9 | |
| | OD | OS | OD | OS | OD | OS | OD | OS |
| SHIRMERS | 4 | 5 | 7 | 8 | 9 | 11 | 8 | 9 |
| TEAR OSMOLARITY | 285 | 285 | 291 | 288 | 296 | 286 | 293 | 281 |
| CORNEAL STAINING | 8 | 8 | 6 | 7 | 5 | 6 | 6 | 5 |
| VISUAL ACUITY | 20/25 | 20/25 | 20/25 + 2 | 20/25 + | 20/20 − 1 | 20/25 + 1 | 20/20 − 1 | 20/25 |
| ARTIFICIAL TEARS USED PER DAY | | V.2 Zero tears used | | V.3 Zero tears used | | V.4 Zero tears used | | |
| 004-DIM | Visit 1 | | Visit 2 | | Visit 3 | | Visit 4 | |
| DOSING DAY | | | | | | | | |
| OSDI | 47.5 | | 20 | | 5 | | 13.6 | |
| | OD | OS | OD | OS | OD | OS | OD | OS |
| SHIRMERS | 16 | 17 | 4 | 3 | 6 | 7 | 3 | 1 |
| TEAR OSMOLARITY | 305 | 295 | 301 | 298 | 291 | 289 | 279 | 328 |
| CORNEAL STAINING | 10 | 11 | 9 | 9 | 8 | 8 | 6 | 6 |
| VISUAL ACUITY | 20/20 | 20/20 | 20/20 | 20/20 | 20/25 | 20/20 | 20/25 | 20/20 |
| ARTIFICIAL TEARS USED PER DAY | | V.2 BID OU | | V.3 QDAY OU | | V.4 QDAY OU | | |
| 005-DLC | Visit 1 | | Visit 2 | | Visit 3 | | Visit 4 | |
| DOSING BID | | | | | | | | |
| OSDI | 79.2 | | 47.5 | | 41.7 | | 20.8 | |
| | OD | OS | OD | OS | OD | OS | OD | OS |
| SHIRMERS | 17 | 10 | 11 | 5 | 11 | 13 | 11 | 3 |
| TEAR OSMOLARITY | 288 | 296 | 280 | 291 | 285 | 290 | 311 | 306 |
| CORNEAL STAINING | 11 | 11 | 9 | 7 | 6 | 7 | 4 | 5 |
| VISUAL ACUITY | 20/40 | 20/40 | 20/25 | 20/25 | 20/30 | 20/25 − 2 | 20/40 | 20/40 |
| ARTIFICIAL TEARS USED PER DAY | | V.2 BID OU | | | V.3 BID OU | V.4 ZERO-BID OU | | |
| 006-EBB | Visit 1 | | Visit 2 | | Visit 3 | | Visit 4 | |
| DOSING QDAY | | | | | | | | |
| OSDI | 15 | | 12.5 | | 6.3 | | 8.3 | |
| | OD | OS | OD | OS | OD | OS | OD | OS |
| SHIRMERS | 7 | 8 | 3 | 5 | 6 | 4 | 10 | 4 |
| TEAR OSMOLARITY | 287 | 285 | 290 | 295 | 290 | 296 | 300 | 301 |
| CORNEAL STAINING | 6 | 8 | 5 | 6 | 7 | 6 | 7 | 6 |
| VISUAL ACUITY | 20/30 | 20/40 − 2 | 20/30 | 20/30 − 2 | 20/30 − 1 | 20/30 − 1 | 20/30 | 20/30 − 2 |
| ARTIFICIAL TEARS USED PER DAY | | V.2 BID OU | | | V.3 BID OU | V.4 BID OU | | |
| 007-JGW | Visit 1 | | Visit 2 | | Visit 3 | | Visit 4 | |
| DOSING BID | | | | | | | | |
| OSDI | 77.3 | | 93.8 | | 81.3 | | 66.7 | |
| | OD | OS | OD | OS | OD | OS | OD | OS |
| SHIRMERS | 9 | 8 | 15 | 12 | 7 | 12 | 10 | 7 |
| TEAR OSMOLARITY | 323 | 310 | 299 | 296 | 295 | 287 | 296 | 290 |
| CORNEAL STAINING | 7 | 7 | 7 | 6 | 6 | 5 | 4 | 4 |
| VISUAL ACUITY | 20/30 − 2 | 20/25 | 20/30 − 2 | 20/30 | 20/30 | 20/30 | 20/30 | 20/25 |
| ARTIFICIAL TEARS USED PER DAY | | V.2 BID OU | | | V.3 BID OU | V.4 BID OU | | |
| 008-JHS | Visit 1 | | Visit 2 | | Visit 3 | | Visit 4 | |
| DOSING QDAY | | | | | | | | |
| OSDI | 25 | | 25 | | 25 | | | |
| | OD | OS | OD | OS | OD | OS | OD | OS |
| SHIRMERS | 11 | 10 | 12 | 13 | 8 | 6 | 3 | 5 |
| TEAR OSMOLARITY | 330 | 321 | 304 | 295 | 291 | 287 | 289 | 288 |
| CORNEAL STAINING | 7 | 7 | 6 | 7 | 7 | 6 | 6 | 6 |
| VISUAL ACUITY | 20/30 | 20/30 | 20/30 | 20/30 − 2 | 20/30 | 20/30 | 20/30 | 20/30 − 2 |
| ARTIFICIAL TEARS USED PER DAY | | V.2 QID-6X DAY OU | | V.3 QID-6X DAY OU | | V.4 6-10 X A DAY OU | | |
| 009-DBF | Visit 1 | | Visit 2 | | Visit 3 | | Visit 4 | |
| DOSING QDAY | | | | | | | | |
| OSDI | 72.9 | | 12.5 | | 6.3 | | 31.3 | |
| | OD | OS | OD | OS | OD | OS | OD | OS |
| SHIRMERS | 1 | 2 | 1 | 1 | 2 | 4 | 1 | 3 |
| TEAR OSMOLARITY | 300 | 305 | 292 | 292 | 290 | 289 | 291 | 290 |
| CORNEAL STAINING | 10 | 10 | 8 | 8 | 8 | 8 | 7 | 7 |
| VISUAL ACUITY | 20/30 − 1 | 20/30 − 2 | 20/30 | 20/30 | 20/30 | 20/30 + 2 | 20/40 + 2 | 20/30 |

| | | | |
|---|---|---|---|
| ARTIFICIAL TEARS USED PER DAY | V.2 BID-TIDOU | V.3 TIDOU | V.4 TID-QID OU |

1. A method of treating dry eye disease comprising:
a) identifying a patient suffering from dry eye disease;
b) obtaining a hemochorial placenta;
c) isolating the amnion from said hemochorial placenta;
d) contacting said amnion with a liquid media in a manner to preserve viability of cellular components of said amnion;
e) applying said contacted liquid media, or portions thereof, to said patient in an amount to treat or ameliorate the effects of dry eye disease.

2. The method of claim 1, wherein said dry eye disease is associated with reduced lacrimal secretions.

3. The method of claim 1, wherein said dry eye disease is associated with hyperosmolarity of lacrimal secretions.

4. The method of claim 1, wherein said dry eye disease is associated with keratitis.

5. The method of claim 1, wherein said dry eye disease is associated with production of inflammatory cytokines.

6. The method of claim 5, wherein said inflammatory cytokines are selected from the group consisting of: a) IL-1 beta; b) IL-6; c) TNF-alpha; and d) IL-15

7. The method of claim 1, wherein said dry eye disease is associated with Meibomian gland dysfunction.

8. The method of claim 1, wherein said liquid media subsequent to contact with said amnion is endowed with one or more ingredients selected from factors and agents that promote any one or more of survival, health, cell attachment and normal differentiation of ocular surface epithelial cells and optionally factors and agents that prevent squamous metaplasia; one or more agents capable of altering the fluid properties of a tear film including at least one agent capable of establishing or maintaining a stable tear film and optionally one or more agents selected from the group consisting of opthalmological lubricating agents, viscosity enhancing agents and agents capable of reducing tear film evaporation.

9. The method of claim 1 wherein said amnion is contacted with said liquid media by means of immersion of said amnion in said liquid media.

10. The method of claim 1, wherein said amnion is cultured in said liquid media at 37 Celsius in a fully humidified atmosphere.

11. The method of claim 1, wherein said liquid media is concentrated and subsequently desalted.

12. The method of claim 1, wherein said liquid media is concentrated by filtration and subsequently desalted.

13. The method of claim 1, wherein said liquid media is concentrated by lyophilization and subsequently desalted.

14. The method of claim 1, wherein said liquid media is used as a source for identifying factors capable of inhibiting dry eye disease.

15. The method of claim 1, wherein said liquid media is applied to the ocular surface.

16. The method of claim 1, wherein said liquid media is used to formulate an ophthalmic composition.

17. The method of claim 16, wherein said ophthalmic composition comprises a bacteriological preservative.

18. The method of claim 17, wherein the bacteriological preservative is selected from the group consisting of benzalkonium chloride, thimerosal, phenylmercuric nitrate, chlorobutanol, and sorbicacid.

19. The method of claim 17, wherein the ophthalmic composition further comprises a chelating agent.

20. The method of claim 17, wherein the ophthalmic composition further comprises an antibiotic.
